(19) United States
(12) Patent Application Publication
(10) Pub. No.: US 2021/0299260 A1
(43) Pub. Date: Sep. 30, 2021

(54) THERAPEUTIC CONJUGATES

(71) Applicant: Ariel Scientific Innovations Ltd., Ariel (IL)

(72) Inventors: Gary GELLERMAN, Rishon-LeZion (IL); Andrii BAZYLEVICH, Ariel (IL)

(73) Assignee: Ariel Scientific Innovations Ltd., Ariel (IL)

(21) Appl. No.: 17/342,608

(22) Filed: Jun. 9, 2021

Related U.S. Application Data
(63) Continuation of application No. PCT/IL2019/051352, filed on Dec. 10, 2019.
(60) Provisional application No. 62/777,267, filed on Dec. 10, 2018.

(51) Int. Cl. A61K 47/54; A61K 47/64; A61K 45/06; C07D 241/08; A61P 35/00

(52) U.S. Cl. CPC A61K 47/545; A61K 47/645; A61P 35/00; C07D 241/08; A61K 45/06

(57) ABSTRACT

Provided herein is a class of anti-cancer conjugates designed to cross the blood-brain barrier (BBB) and thereby deliver a methylation agent into cancerous tumor cells, wherein the conjugate comprises a BBB-shuttle moiety that allows the conjugate to cross the BBB, and at least two methylation moieties of the methyltriazene type attached thereto for effecting DNA methylation in the cancerous cells. Also provided are uses of the conjugates in the treatment of brain cancer.

### RELATED APPLICATIONS

This application is a Continuation of PCT Patent Application No. PCT/IL2019/051352 having international filing date of Dec. 10, 2019, which claims the benefit of priority under USC § 119(e) of U.S. Provisional Patent Application No. 62/777,267 filed on Dec. 10, 2018. The contents of the above applications are all incorporated by reference as if fully set forth herein in their entirety.

### FIELD AND BACKGROUND OF THE INVENTION

The present invention, in some embodiments thereof, relates to a class of therapeutic conjugates, and more particularly, but not exclusively, to compounds capable of drug delivery of a bioactive agent through the blood-brain barrier.

Brain cancer treatment is still one of the biggest challenges in oncology. Three major types of brain tumors are recognized by the World Health Organization, as a classification of gliomas: astrocytomas, oligodendrogliomas, and oligo-astrocytomas. These tumors are further classified by subtypes (mainly for astrocytomas) and are graded from Ito IV based on histology with grade IV being the most aggressive glioblastoma multiforme (GBM). Malignant astrocytomas constitute about 50-60% of primary brain tumors, with a peak incidence in the fifth or sixth decade of life that ranges from 5 to 8 per 100,000 inhabitants. The incidence of brain tumors seems to be increasing, but it is unclear if this is due to environmental or genetic factors.

The standard treatment for brain tumors consists of maximal surgical resection, followed by radiotherapy and chemotherapy. However, despite continued research and new approaches, the prognosis for patients with malignant brain tumors is still extremely poor. Thus, the median survival of patients with GBMs is only 20 weeks by surgical resection alone, 36 weeks by surgery and radiation, and inclusion of standard cytotoxic chemotherapy offers a minimal survival advantage, raising the median survival to 40-50 weeks.

In the last decades, despite advances in anticancer drug discovery and development, there has been little improvement on the prognosis of patients with brain cancer. Often, it has been found that promising agents for primary brain cancers in vitro have had little impact on disease in clinical trials. These disappointing results can be at least in part explained by the inability to deliver therapeutic agents to the CNS across the blood-brain barrier (BBB) avoiding various resistance mechanisms and to reach the desired targets. Moreover, it should be also taken into account that low-molecular weight chemotherapeutics do not achieve and maintain effective steady state concentrations within malignant glioma cells because of short blood half-lives.

A review by Laquintana, V. et al. [Expert Opin Drug Deliv., 2009, 6(10) pp. 1017-1032] focuses on strategies for delivering anticancer drugs to the CNS by chemical modification of drugs as well as by designing efficient targeted vectors (such as antibodies and protein carriers) or nanosystems (colloidal carriers) able to cross biological barriers as BBB in a controlled and non-invasive manner.

Temozolomide (TMZ), a DNA metylation drug of the triazene class, is still the standard in treatment of GBM. TMZ is a small (194 Da) lipophilic molecule (see scheme below), that is administrated orally, and releases upon degradation the active methyl cation (Met) that subsequently methylates purine bases of DNA.

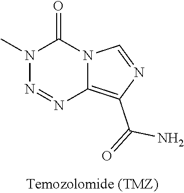

Despite its limited impact on overall survival of the patients, TMZ is still the most used drug in chemotherapy against GBM. To improve the efficacy of TMZ and reduce the side effects of chemotherapy, systemic TMZ administration using a biodegradable carrier such as nanoparticles and conjugates with chitosan and biotin were widely studied. Yet only limited clinical benefit from these studies was achieved.

Waldeck, W. et al. [Int J Med Sci, 2008, 5(5), pp. 273-284] present a study of the pharmacologic potency with simultaneous reduction of unwanted adverse reactions of the highly efficient chemotherapeutic temozolomide (TMZ) as an example. The TMZ connection to transporter molecules (TMZ-BioShuttle) resulted in a much higher pharmacological effect in glioma cell lines while using reduced doses. The re-formulation of TMZ to TMZ-BioShuttle achieved a nearly 10-fold potential of the established pharmaceutic TMZ far beyond the treatment of brain tumors cells and results in an attractive reformulated drug with enhanced therapeutic index.

### SUMMARY OF THE INVENTION

As discussed hereinabove, glioblastoma multiforme (GBM) is the most common and aggressive brain tumor with limited remedies and prognosis, and temozolomide (TMZ), the DNA methylation drug, is still the standard in treatment of this disease. The present disclosure presents a molecular conjugate useful in the treatment of GBM. The presently disclosed conjugate comprises a BBB-permeable shuttle, such as N,N-dimethyl-diketopiperazine (DKP) or tetra-N(Me)Val or Phe peptidyls, and the DNA methylation dimethyltriazene. As presented hereinbelow, survival study on A172 (GBM) cell line xenograft model, the exemplary embodiment DKP-dimethyltriazene conjugate showed superior activity compared to TMZ in overall survival, exhibiting no weight loss even at elevated dose of 50 mg/kg. Histopathology analysis of mice brains treated with the exemplary conjugate pointed to prominent reduction in tumor area, tumor distribution and normal brain structure related to brain sections, while in TMZ treated mice both lateral and third ventricles were significantly enlarged evidencing CSF retention and appearance of hydrocephalus pathology.

Aspects of the present invention are drawn to a family of compounds, also referred to herein as “conjugates”, that share the common property of exhibiting a blood-brain barrier shuttle moiety, and at least two methylation moieties, each independently represented by general Formula I:

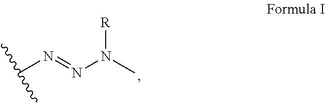

wherein R is selected from the group consisting of hydrogen, a C₁₋₄ alkyl, a labeling moiety, and a bioactive agent. In some embodiments, the labeling moiety is a light absorbing moiety, a light emitting moiety, a magnetic moiety, or an isotope-containing moiety. In some embodiments, the bioactive agent is a drug, a reported molecule or a receptor ligand.

According to some embodiments of the invention, the blood-brain barrier shuttle moiety is a small molecule or an oligopeptide.

According to some embodiments of the invention, the small molecule is selected from the group consisting of 1,4-dialkyl-3,6-diarylpiperazine-2,5-dione, 1,4-dimethyl-3,6-diarylpiperazine-2,5-dione (DKP), diaza diketocyclooctane, and N,N′-diaza diketocyclooctane.

According to some embodiments of the invention, the compound disclosed herein is represented by general Formula II:

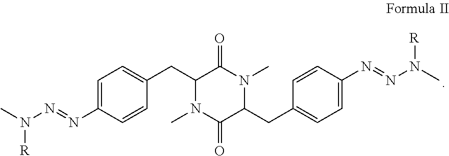

According to some embodiments of the invention, the compound disclosed herein is (3S,6S)-1,4-dimethyl-3,6-bis(4-((E)-3-methyltriaz-1-en-1-yl)benzyl)piperazine-2,5-dione or (3S,6S)-3,6-bis(4-((E)-3,3-dimethyltriaz-1-en-1-yl)benzyl)-1,4-dimethylpiperazine-2,5-dione (Compound 1).

According to some embodiments of the invention, the oligopeptide BBB-shuttle moiety is selected from the group consisting of (N-Me-Phenylalanine)ₙ, (N-Me-Tryptophan)ₙ, (N-Me-1-Naphthylalanine)ₙ, (N-Me-2-Naphthylalanine)ₙ, (N-Me-Tyrosine)ₙ and (N-Me-DOPA)ₙ, whereas n is an integer equal or greater than 2.

According to some embodiments of the invention, the methylation moieties is attached to a side-chain of at least one of the amino-acids of the oligopeptide BBB-shuttle moiety.

According to some embodiments of the invention, the labeling moiety or the bioactive agent is attached to the methylation moiety via a labile linking moiety.

According to another aspect of embodiments of the present invention, there is provided a process of preparing the conjugate presented herein, essentially as described hereinbelow.

According to another aspect of embodiments of the present invention, there is provided a pharmaceutical composition that includes the conjugates provided herein.

According to some embodiments of the invention, the pharmaceutical composition is packaged and labeled for use in the treatment of cancer.

According to some embodiments of the invention, the pharmaceutical composition is packaged and labeled for use in diminishing brain tumors.

According to another aspect of embodiments of the present invention, there is provided a use of the conjugates provided herein in the preparation of a medicament.

According to some embodiments of the invention, the medicament is for treating cancer. In some embodiments, the medicament of for treating brain cancer.

According to an aspect of the present invention, there is provided a method of treatment of cancer, the method includes administering to a subject in need thereof, a therapeutically effective amount of at least one the conjugates provided herein.

According to yet another aspect of embodiments of the present invention, there is provided a building-block compound that includes an amino acid and a methylation moiety attached to a side-chain of the amino acid, and represented by general Formula III:

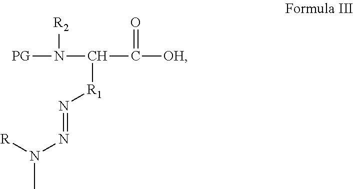

wherein:

R is selected from the group consisting of hydrogen, a C₁₋₄ alkyl, a labeling moiety, and a bioactive agent,

R₁ is the side-chain,

R₂ is a hydrogen or a C₁₋₄ alkyl, and

PG is hydrogen or a protecting group.

According to some embodiments, R₁ in the building-block compound is selected from the group consisting of a branched or unbranched, substituted or unsubstituted alkyl interrupted or uninterrupted by one or more heteroatom, a substituted or unsubstituted aryl, and a substituted or unsubstituted heteroaryl.

According to some embodiments, R₁ is a p-, m- or o-substituted phenyl, R₂ is hydrogen, methyl or ethyl, R is hydrogen or methyl, and PG is hydrogen or an α-amino protecting group.

According to some embodiments, the building-block compound is Compound 9 as set forth hereinabove.

According to some embodiments, the labeling moiety or the bioactive agent in the building-block compound is attached to the methylation moiety via a labile linking According to yet another aspect of embodiments of the present invention, there is provided an oligopeptide that is a BBB-shuttle and that includes at least one of the According to some embodiments, the oligopeptide includes at least two residues of the building-block compounds disclosed herein.

According to some embodiments, the oligopeptide is (2R,5S,8R,11S)-2,8-bis(4-((E)-3,3-dimethyltriaz-1-en-1-yl)benzyl)-5,11-diisopropyl-3,6,9,12-tetramethyl-4,7,10,13 -tetraoxo-3,6,9,12-tetraazatetradecanoic acid (Compound 19).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D present the results of stability and degradation assays conducted for Compound 1 in PBS (FIG. 1A), acetate buffer (FIG. 1B), Hep G2 cell line (FIG. 1C): medium vesus cell lysate, and the detection of the metabolite (E)-3-(4-aminobenzyl)-1,4-dimethyl-6-(4-(3-methyltriaz-1-en-1-yl)benzyl)piperazine-2,5-dione (see, scheme below) in Hep G2 cell line lysate by measurement of mass peak area (LCMS) (FIG. 1D);

FIG. 2 presents intracranial tumor-bearing mice weight gain, wherein the plot of the mice treated with Compound 1 is marked with red circles, plot of mice treated with TMZ is marked with blue circles, and plot of the control group is marked by black circles (GraphPad Prism 5; and “***” p<0.001);

FIGS. 3A-B present the results obtained in the survival rate assays (FIG. 3A), and mice appearance on termination day (FIG. 3B.); and

FIG. 4 presents GBM tumor presence and distribution in brain sections stained with H&E, wherein three slides within a distance of 100 μm apart represent one mice from each experimental group.

### DESCRIPTION OF SOME SPECIFIC EMBODIMENTS OF THE INVENTION

The present invention, in some embodiments thereof, relates to a class of therapeutic conjugates, and more particularly, but not exclusively, to compounds capable of drug delivery of a bioactive agent through the blood-brain barrier.

The principles and operation of the present invention may be better understood with reference to the figures and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

As presented hereinabove, some of the challenges still to overcome in brain tumor eradication treatment using methylation agents, relate to the poor pharmacokinetic properties of presently know drugs, such as TMZ. TMZ is labile above pH 7 with a plasma half-life of 1.8 hours at pH 7.4 and acts similarly to another DNA methylating triazene drug—dacarbazine (DTIC):

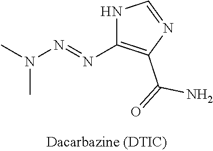

wherein upon spontaneous breakdown, TMZ forms monomethyl triazene metabolite 5-(3-methyltriazen-1-yl)-imidazole-4-carboxamide (MTIC):

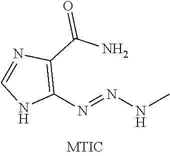

and MTIC further reacts with water to release 5-aminoimidazole-4-carboxamide (AIC) and the highly reactive methyldiazonium cation.

The active agent methyldiazonium cation preferably methylates DNA at N7 positions of guanine in guanine rich regions (N7-MeG; 70%), but also methylates N3 adenine (N3-MeA; 9%) and O6 guanine residues (O6-MeG; 6%). There is a narrow pH window close to physiological pH at which the whole process of TMZ prodrug activation to methyl cation transfer can occur. Brain tumors possess a more alkaline pH compared with surrounding healthy tissue, a situation which favors TMZ activation preferentially within tumor tissue.

In general, bioactive molecules can be efficiently delivered to the brain utilizing BBB shuttles as carriers. Exemplary BBB-shuttle moieties, such as DKP and (N-MePhe)_{4,} have been successfully applied in delivery of several cargo moieties, such as short peptides, GABA, Nip and ALA. These shuttles act through transcellular passive diffusion, which is characteristic of small lipophilic compounds.

While conceiving the present invention, the inventors contemplated conjugating a dimethyl triazene moiety, which is the therapeutic portion of non-BBB-permeable dacarbazine, with a BBB shuttling agent diketopiperazine (DKP):

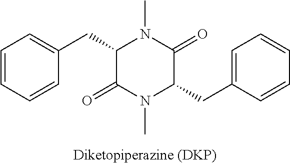 [SMILES: CN1C(=O)[C@H](Cc2ccccc2)N(C)C(=O)[C@@H]1Cc1ccccc1]

aiming to achieve efficient delivery of the DNA methylation agent through BBB, using the BBB-shuttling properties of DKP. The other advantage that had been contemplated by the inventors, is the intensified DNA methylation capabilities of the resulting conjugate (see Examples section below) due to the presence of two DNA methylation equivalents per one DKP molecule, which may lead to increased concentration of Me+ cations at the target. While reducing the present invention to practice, a conjugate that consists of the known BBB-shuttle di-[N-methyl Phe] DKP covalently linked to two Me+ releasing dimethyltriazene moieties at the phenyl rings has been prepared and tested.

Therapeutic Conjugate:

Thus, according to an aspect of some embodiments of the present invention, there is provided a compound, which is a conjugate between at least two methylation moieties and a moiety that is capable of crossing the blood-brain barrier and transport the methylation moieties across the BBB. In other words, the compound according to some embodiments of the present invention, is a BBB-shuttle having at least two methylation moieties attached thereto, which is capable of, upon administration to a subject having brain tumors, effecting DNA methylation in cancerous cells in the brain tumors and also elsewhere in the central nervous system (CNS). The compound is also capable of effecting methylation of other substrates beside DNA in other environments beside brain tumors.

Methylation Moiety:

It is noted herein that the methylation moiety, forming a part of the compounds presented herein, can be of the type that becomes active spontaneously in physiological conditions, or the type that requires some form of enzymatic activation, such as effected by the hepatic system.

According to some embodiments of the present invention, the compound provided herein includes at least two methylation moieties, each independently represented by general Formula I:

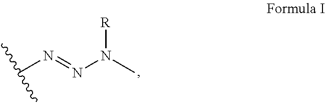

wherein R is selected from the group consisting of hydrogen, a C₁₋₄ alkyl, a labeling moiety, a bioactive agent and any other functional moiety useful in any specific intended use of the compound.

The term “alkyl” describes a saturated aliphatic hydrocarbon including linear chains (unbranched) and branched aliphatic hydrocarbons. Preferably, the alkyl group has 1 to 4 carbon atoms (C₁₋₄ alkyl or low alkyl), or 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is low alkyl having 1-4 carbons, or a medium size alkyl having 4 to 10 carbon atoms. Most preferably, unless otherwise indicated, the alkyl is a lower alkyl having 1 to 4 carbon atoms. The alkyl group may be substituted or unsubstituted. Substituted alkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine.

The alkyl group can be an end group, wherein it is attached to a single adjacent atom, or a linking moiety, which connects two or more moieties via at least two carbons in its chain. When an alkyl is a linking moiety, it is also referred to herein as “alkylene”, e.g., methylene, ethylene, propylene, etc.

The term “alkenyl” describes an unsaturated alkyl, as defined herein, having at least two carbon atoms and at least one carbon-carbon double bond. The alkenyl may be substituted or unsubstituted by one or more substituents, as described for alkyl hereinabove.

The terms “alkynyl” or “alkyne”, as defined herein, is an unsaturated alkyl having at least two carbon atoms and at least one carbon-carbon triple bond. The alkynyl may be substituted or unsubstituted by one or more substituents, as described hereinabove.

In the 3-substituted-3-methyltriazene-1-yl group, constituting the methylation moiety, according to some embodiments of the present invention, R can be a labeling moiety, a bioactive agent and any other functional moiety useful in any specific intended use of the compound. The term “labeling moiety”, as used herein refers to a moiety that emits or absorbs a signal that can be detected by an instrument, thereby be used to report the presence and in some cases the amount of a compound bearing this moiety in various environments.

In the context of the present embodiments, the terms “bioactive agent”, “pharmaceutically active agent” and “drug” are used interchangeably.

As used herein, the terms “bioactive agent” and “drug” refer to small molecules or biomolecules that alter, inhibit, activate, or otherwise affect a biological mechanism or event. Bioactive agent that can be tethered to the compound presented herein, according to embodiments of the present invention, include, but are not limited to, anti-cancer substances for all types and stages of cancer and cancer treatments (chemotherapeutic, proliferative, acute, genetic, spontaneous etc.), anti-proliferative agents, chemosensitizing agents, anti-inflammatory agents (including steroidal and non-steroidal anti-inflammatory agents and anti-pyretic agents), antimicrobial agents (including antibiotics, antiviral, antifungal, anti-parasite, anti-protozoan etc.), anti-oxidants, hormones, anti-hypertensive agents, anti-AIDS substances, anti-diabetic substances, immunosuppressants, enzyme inhibitors, neurotoxins, opioids, hypnotics, anti-histamines, lubricants, tranquilizers, anti-convulsants, muscle relaxants and anti-Parkinson substances, antipruritic agents, anti-spasmodics and muscle contractants including channel blockers, miotics and anti-cholinergics, anti-glaucoma compounds, modulators of cell-extracellular matrix interactions including cell growth inhibitors and anti-adhesion molecules, vitamins, vasodilating agents, inhibitors of DNA, RNA or protein synthesis, analgesics, anti-angiogenic factors, anti-secretory factors, anticoagulants and/or anti-thrombotic agents, anesthetics, ophthalmics, prostaglandins, anti-depressants, anti-psychotic substances, anti-emetics, radioactive agents and imaging agents. A more comprehensive listing of exemplary drugs suitable for use in the present invention may be found in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, edited by Susan Budavari et al., CRC Press, 1996, and the United States Pharmacopeia-25/National Formulary-20, published by the United States Pharmcopeial Convention, Inc., Rockville Md., 2001.

Anti-cancer drugs that can be linked and controllably released from the molecular structure according to some embodiments of the invention include, but are not limited to Chlorambucil; 3-(9-Acridinylamino)-5-(hydroxymethyl)aniline; Azatoxin; Acivicin; Aclarubicin; Acodazole Hydroclhloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta- I a; Interferon Gamma- I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

Non-limiting examples of chemotherapeutic agents that can be efficiently delivered by the molecular structures of the present invention, include amino containing chemotherapeutic agents such as camptothecin, daunorubicin, doxorubicin, N-(5,5-diacetoxypentyl)doxorubicin, anthracycline, mitomycin C, mitomycin A, 9-amino aminopertin, antinomycin, N⁸-acetyl spermidine, 1-(2-chloroethyl)-1,2-dimethanesulfonyl hydrazine, bleomycin, tallysomucin, and derivatives thereof; hydroxy containing chemotherapeutic agents such as etoposide, irinotecan, topotecan, 9-amino camptothecin, paclitaxel, docetaxel, esperamycin, 1,8-dihydroxy-bicyclo[7.3.1] trideca-4-ene-2,6-diyne-13-one, anguidine, morpholino-doxorubicin, vincristine and vinblastine, and derivatives thereof, sulfhydril containing chemotherapeutic agents and carboxyl containing chemotherapeutic agents. Additional chemotherapeutic agents include, without limitation, an alkylating agent such as a nitrogen mustard, an ethylenimine and a methylmelamine, an alkyl sulfonate, a nitrosourea, and a triazene; an antimetabolite such as a folic acid analog, a pyrimidine analog, and a purine analog; a natural product such as a vinca alkaloid, an epipodophyllotoxin, an antibiotic, an enzyme, a taxane, and a biological response modifier; miscellaneous agents such as a platinum coordination complex, an anthracenedione, an anthracycline, a substituted urea, a methyl hydrazine derivative, or an adrenocortical suppressant; or a hormone or an antagonist such as an adrenocorticosteroid, a progestin, an estrogen, an antiestrogen, an androgen, an antiandrogen, a gonadotropin-releasing hormone analog, bleomycin, doxorubicin, paclitaxel, 4-OH cyclophosphamide and cisplatinum.

According to some embodiments of the present invention, the labeling moiety or the bioactive agent is attached to the methylation moiety via a labile linking moiety. According to some embodiments, the labile linking moiety is selected from the group consisting of:

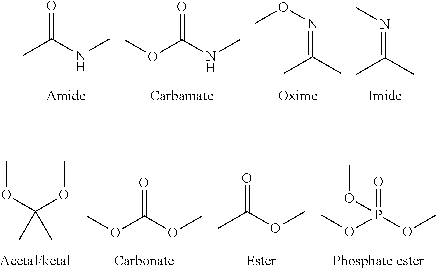

Definitions of specific functional groups, chemical terms, and general terms used throughout the specification are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987.

BBB-Shuttle:

According to some embodiments of the present invention, the blood-brain barrier shuttle (BBB-shuttle) moiety is a small molecule or an oligopeptide. In the context of embodiments of the present invention, a BBB-shuttle is a molecular entity that can diffuse through the BBB under physiological conditions, or be transported through the BBB by any other molecular mechanism. A BBB-shuttle is therefore a molecular vector that can be administered to a subject orally or intravenously, or in any other mode of administration that gets the molecular entity into the systemic blood stream, and find its way into the CNS. A moiety that is attached to the BBB-shuttle is regarded as the cargo of the shuttle, and the cargo's destination is typically a CNS organ, such as the brain.

A selection of a BBB-shuttle moiety for the compound provided herein is generally within the capacity of a person skilled in the relevant art, based on the accumulated knowledge in the published literature. Information regarding BBB-shuttles suitable for use as a blood-brain barrier shuttle moiety, according to embodiments of the present invention, can be found, for example, in Malakoutikhah, M. et al., “Shuttle-mediated drug delivery to the brain”, Angew Chem Int Ed Engl., 2011, 50(35), pp. 7998-8014; Banks, Wash., “Characteristics of compounds that cross the blood-brain barrier”, BMC Neurol., 2009, 1, S3; and Sánchez-Navarro, M. et al., “Blood-brain barrier peptide shuttles”, Curr Opin Chem Biol., 2017, 38, pp. 134-140. The skilled artisan would appreciate the information provided in the published literature regarding BBB-shuttle moieties that can be modified to carry a specific cargo or be modified so as to exhibit a methylation moiety, as described herein.

According to some embodiments of the present invention, non-limiting examples of BBB-shuttles of the “small molecule” category include 1,4-dialkyl-3,6-diarylpiperazine-2,5-dione. 1,4- dimethyl-3,6-diarylpiperazine-2,5-dione (DKP), diaza diketocyclooctane, and N,N′-diaza diketocyclooctane.

An exemplary non-limiting examples of the conjugate compound, according to some embodiments of the present invention, based on a small molecule BBB-shuttle, is presented in the Examples section that follows below in the form of Compound 1 and its single-methyl derivative, both represented by general Formula II:

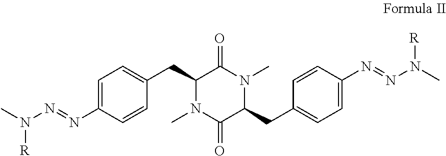

wherein R is hydrogen in the mono-methyl case, and R is a methyl in the case of Compound 1.

According to some embodiments of the present invention, non-limiting examples of BBB-shuttles of the “oligopeptide” category include R and/or S stereo-configuration of natural and unnatural amino acid building blocks oligopeptides, (N-Me-Phenylalanine)ₙ, (N-Me-Tryptophan)ₙ, (N-Me-1-Naphthylalanine)ₙ, (N-Me-2-Naphthylalanine)ₙ, (N-Me-Tyrosine)ₙ and (N-Me-DOPA)ₙ, whereas n is an integer equal or greater than 2, and more preferably from 2 to 10.

Methylation Oligopeptide Building-Block Unit:

According to some embodiments of the present invention, at least one of the methylation moieties is attached to a side-chain of at least one of the amino-acids of an oligopeptide.

According to an aspect of some embodiments of the present invention, the conjugate is in the form of an oligopeptide that comprises, as a main-chain building-block (unit), a residue of an amino acid having a methylation moiety attached to the side-chain thereof.

Hence, there is provided a compound in the form of an amino-acid, which is represented by general Formula III:

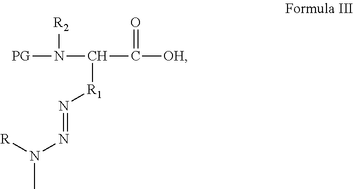

wherein:

R is selected from the group consisting of hydrogen, a C₁₋₄ alkyl, a labeling moiety, and a bioactive agent,

R₁ is said side-chain,

R₂ is a hydrogen or a C₁₋₄ alkyl or a labeling moiety, or a bioactive agent, and

PG is hydrogen or a protecting group.

In the context of embodiments of the present invention, the amino acid includes a side-chain residue on which a methylation moiety can be attached. The side-chain residue is denoted R₁ in Formula II and can be a branched or unbranched, substituted or unsubstituted alkyl interrupted or uninterrupted by one or more heteroatom, a substituted or unsubstituted aryl, and a substituted or unsubstituted heteroaryl, as these terms are defined herein or known in the art.

In Formula III, PG represents a hydrogen atom or a protecting group, as this term is known and used in the art of peptide chemistry. For more information pertaining to protecting groups and amino acid protection, the use can refer to the published literature, such as Isidro-Llobet, A. et al., “Amino acid-protecting groups”, Chem Rev., 2009, 109(6), pp. 2455-504.

In one exemplary embodiment, the amino acid residue is a derivative of phenylalanine, namely R₁ is a p-, m- or o-substituted phenyl, R₂ is hydrogen, methyl or ethyl, R is hydrogen or methyl, and PG is hydrogen or an α-amino protecting group.

A more specific example of an oligopeptide building-block unit suitable for constructing a conjugate of a methylation agent and a BBB-shuttle, according to some embodiments of the present invention, is Compound 9, the synthesis and use of it is presented in the Examples section that follows below:

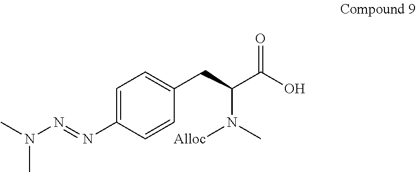

In addition to the methylation moiety, the amino-acid building-block may further include a labeling moiety or a bioactive agent, which may optionally be attached to the building-block on the methylation moiety or the main-chain amino group via a labile linking moiety, as these terms are defined herein.

It is to be understood that the present disclosure encompasses the amino-acid building-block unit by itself, as well as any oligopeptide that includes one residue or more of such building-block unit. A non-limiting example of such an oligopeptide is demonstrated in the Examples section that follows below, in the form of Compound 19 or (2R,5S,8R,11S)-2,8-bis(4-((E)-3,3-dimethyltriaz-1-en-1-yl)benzyl)-5,11-diisopropy1-3,6,9,12-tetramethyl-4,7,10,13-tetraoxo-3,6,9,12-tetraazatetradecanoic acid.

Uses and Applications:

The conjugate compound presented herein can be used as an active ingredient in a pharmaceutical composition or a medicament, particularly for treating cancer, and more specifically, brain cancer. In general, the conjugate compounds provided herein are preferably useful in treating medical conditions in which malignant brain tumors are involved. In some embodiments of the present invention, the medical condition is associated with malignant cells and tumors, collectively referred to herein as cancer.

To date, chemotherapy remains the most common and most frequently used in cancer treatment, alone or in combination with other therapies. Currently available anticancer chemotherapies act by affecting specific molecular targets in proliferating cancer cells, leading to inhibition of essential intracellular processes such as DNA transcription, synthesis and replication. The use of a conjugate according to embodiments of the present invention, can optimize the balance between the desired anticancer activity of certain anticancer drugs and their adverse side effects, by quantitative determination of the actual amount of drug released in the targeted cells.

In some embodiments, the targeting moiety of the conjugates presented herein, is responsible for the higher concentration of the conjugate at the targeted bodily site compared to non-targeted bodily sites, thereby reducing the adverse side effects associated with the toxicity of the anti-cancer drugs attached thereto. In addition, the linking moieties attached the anti-cancer drugs to the conjugate are selected such that they cleave in conditions that are present at the targeted site more so than in non-targeted sites, thereby releasing the payload of drugs at the targeted site at a higher rate compared to non-targeted sites.

It is expected that during the life of a patent maturing from this application many relevant ratiometric luminescent theranostic conjugates will be developed and the scope of the term ratiometric luminescent theranostic conjugates is intended to include all such new technologies a priori.

As used herein the term “about” refers to±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the phrases “substantially devoid of” and/or “essentially devoid of” in the context of a certain substance, refer to a composition that is totally devoid of this substance or includes less than about 5, 1, 0.5 or 0.1 percent of the substance by total weight or volume of the composition. Alternatively, the phrases “substantially devoid of” and/or “essentially devoid of” in the context of a process, a method, a property or a characteristic, refer to a process, a composition, a structure or an article that is totally devoid of a certain process/method step, or a certain property or a certain characteristic, or a process/method wherein the certain process/method step is effected at less than about 5, 1, 0.5 or 0.1 percent compared to a given standard process/method, or property or a characteristic characterized by less than about 5, 1, 0.5 or 0.1 percent of the property or characteristic, compared to a given standard.

The term “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The words “optionally” or “alternatively” are used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the terms “process” and “method” refer to manners, means, techniques and procedures for accomplishing a given task including, but not limited to, those manners, means, techniques and procedures either known to, or readily developed from known manners, means, techniques and procedures by practitioners of the chemical, material, mechanical, computational and digital arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental and/or calculated support in the following examples.

### EXAMPLES

Reference is now made to the following examples, which together with the above descriptions illustrate some embodiments of the invention in a non-limiting fashion.

### Example 1

### Synthesis of a BBB-Permeable Methylation Conjugate (Compound 1)

Provided herein is an exemplary synthetic route for affording the conjugate (3S,6S)-3,6-bis(4-((E)-3,3-dimethyltriaz-1-en-1-yl)benzyl)-1,4-dimethylpiperazine-2,5-dione (Compound 1, see scheme below).

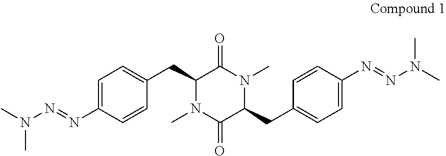

One optional synthesis is described below and includes several synthetic steps, as presented in Scheme 1 below. For initiation of the synthesis, the chosen starting material p-nitro-L-Phe, was subjected first to N-Boc protection by Boc₂O, yielding Compound 7 according to a procedure described in the literature.

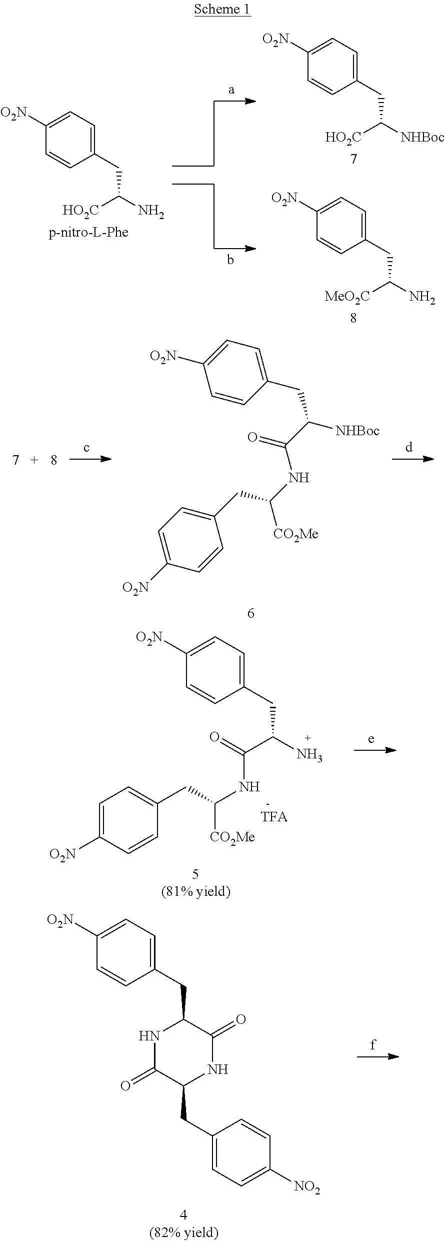 [SMILES: N[C@@H](Cc1ccc([N+](=O)[O-])cc1)C(=O)O ; CC(C)(C)OC(=O)N[C@@H](Cc1ccc([N+](=O)[O-])cc1)C(=O)O ; COC(=O)[C@@H](N)Cc1ccc([N+](=O)[O-])cc1 ; COC(=O)[C@H](Cc1ccc([N+](=O)[O-])cc1)NC(=O)[C@H](Cc1ccc([N+](=O)[O-])cc1)NC(=O)OC(C)(C)C ; COC(=O)[C@H](Cc1ccc([N+](=O)[O-])cc1)NC(=O)[C@@H](N)Cc1ccc([N+](=O)[O-])cc1 ; O=C1N[C@@H](Cc2ccc([N+](=O)[O-])cc2)C(=O)N[C@H]1Cc1ccc([N+](=O)[O-])cc1]

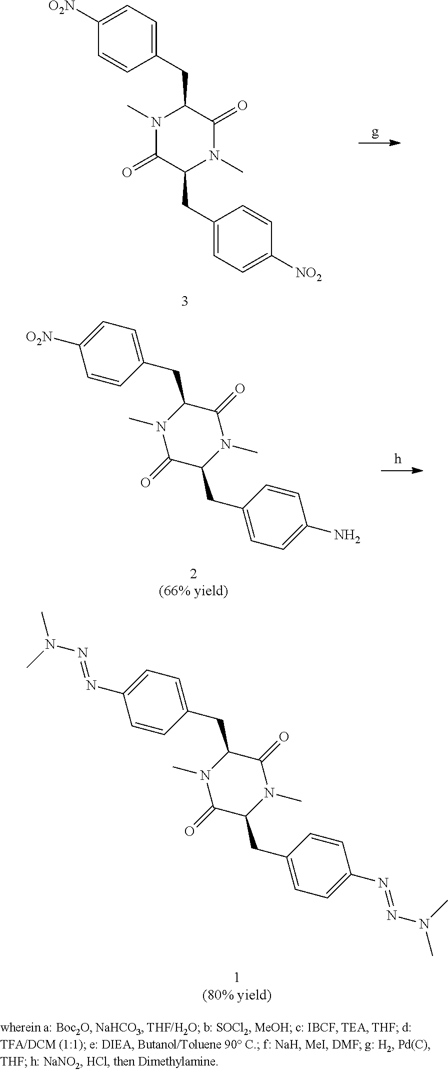

In parallel, Boc-4-nitro-Phe was esterified leading to 4-nitro-Phe methyl ester. Coupling the reaction between Compound 7 and Compound 8 (isobutyl chloroformate, TEA) led to Compound 6 that was subjected to further Boc deprotection (TFA/DCM 1:1), resulting in deprotected intermediate Compound 5, collected by filtration after precipitation in cold diethyl ether (81% yield). Further, Compound 5 was cyclized to diketopiperazine Compound 4 (82% yield) by heating in a mixture of toluene/n-butanol (1:1) for 6 hours in the presence of DIPEA. The afforded diketopiperazine was subsequently dimethylated (NaH, CH₃I) leading to intermediate Compound 3 (not isolated), which was further subjected to reduction of nitro groups (H_{2,} Pd/C in THF), giving dianiline intermediate Compound 2 at 66% yield. The final step included first double diazotization of Compound 2 in 37% HCl with NaNO₂ under cooling followed by gentle addition of large excess of aqueous dimethyl amine. Finally, purification of the crude product on silica by flash chromatography afforded the desired Compound 1 in 89% yield.

Synthesis of (S)-methyl 2-((5)-2-amino-3-(4-nitrophenyl)propanamido)-3-(4-nitrophenyl)propanoate (Compound 5): Boc-4-nitrophenylalanine 5 g, (16.13 mmol) was dissolved in dry THF, followed by addition of TEA 2.25 ml (16.13 mmol). The mixture was cooled to 0° C. and isobutyl chloroformate was added dropwise. The mixture was stirred 10 min at 0° C. (white deposition was formed). Then the solution of Boc-4-nitro-Phe methyl ester (16.13 mmol) and TEA in dry THF was added to the mixture and stirred overnight at r.t. After completion of reaction the mixture was diluted with ice water and white deposition was washed with water 3 times, saturated sodium carbonate, citric acid and again with water 3 times. Obtained white powder was dried and dissolved in cold solution of TFA/DCM (1:1). The mixture was stirred 1.5 hours, evaporated, and cold diethyl ether was added leading to precipitation of 6.94 g (13.08 mmol) of pure Compound 5 (81% yield). ¹H NMR (400 MHz, DMSO-d6) δ ppm: 9.09 (d, J=7.7Hz, 1 H), 8.28 (br. s., 2H), 8.13-8.22 (m, 4H), 7.45-7.58 (m, 4H), 4.70 (m, 1H), 4.13 (m, 1H), 3.60-3.66 (m, 3H), 3.17-3.28 (m, 2H), 3.06-3.16 (m, 2H). ¹³C NMR (100 MHz, DMSO-d6): δ 170.4 (C-15), δ 167.6 (C-12), δ 146.6 (C-1), δ 146.2 (C-25), δ 144.7 (C-4), δ 142.6 (C-22), δ 130.7 (C-2), δ 130.7 (C-6), δ 130.3 (C-24), δ 130.3 (C-26), δ 123.2 (C-23), δ 123.2 (C-27), δ 123.1 (C-3), δ 123.1 (C-5), δ 52.8 (C-10), δ 52.5 (C-18), δ 52.0 (C-7), δ 36.3 (C-21), δ 36.0 (C-9). HRMS m/z (EI⁺) C₁₉H₂₀N₄O₇ calculated [M+H]⁺417.1405; found 417.1426.

Synthesis of (3S,6S)-3,6-bis(4-nitrobenzyl)piperazine-2,5-dione (Compound 4): Compound 5 5 g (9.43 mmol) was heated with DIPEA in toluene/butanol-1 mixture during 6 h at 90° C. Deposition was filtered, washed with cold ethyl acetate and dried overnight. Compound 4 was obtained as a gray powder, 2.97 g (7.73 mmol, 82% yield). ¹H NMR (400 MHz, DMSO-d6) δ ppm: 8.25 (s, 2H), 8.02 (d, J=8.9Hz, 4H), 7.27 (d, J=8.9Hz, 4H), 4.18-4.29 (m, 2H), 2.69 -2.92 (m, 4H). ¹³C NMR (100 MHz, DMSO-d6): δ 166.0 (C-3), δ 166.0 (C-6), δ 145.8 (C-19, 11), δ 144.4 (C-16, 8), δ 130.8 (C-10, 12, 20, 18), δ 122.6 (C-9, 13, 21, 17), δ 54.4 (C-2, 5), δ 37.34 (C-7, 15). HRMS m/z (EI⁺) C₁₈H₁₆N₄O₆ calculated [M+H]⁺385.1143; found: 385.1159.

Synthesis of 3,6-bis(4-aminobenzyl)-1,4-dimethylpiperazine-2,5-dione (Compound 3): Compound 4, 2 g (5.20 mmol) was suspended in 10 ml of dry DMF and cooled at ice bath under inert atmosphere. Sodium hydride, 416 mg (60% in mineral oil) was added and mixture was stirred 10 minutes. Methyl iodide, 647 μL (10.40 mmol) was added and mixture was stirred additional 10 min at 0° C. The reaction mixture was poured in ice water; the precipitate was collected, washed with water, dried, suspended in THF and placed to the hydrogenator with addition of 0.2 g of Pd/C (10%). Hydrogenation was performed at 2 atm during 1 hour. After completion of reduction, solution was filtered, evaporated and separated on silica giving Compound 3 1.21 g (3.43 mmol 66% yield). ¹H NMR (400 MHz, DMSO-d6) δ ppm: 6.71 (d, J=8.3Hz, 4H), 6.50 (d, J=8.3Hz, 4H), 6.50 (d, J=8.3Hz, 4H), 4.91 (s, 4H), 3.99 (dd, J=6.1, 4.46Hz, 2H), 2.63 (s, 6H), 2.59 (dd, J=14.3, 4.2Hz, 2H), 2.09 (dd, J=14.1, 6.3Hz, 2H). ¹³C NMR (100 MHz, DMSO-d6): δ 164.8 (C-6), δ 164.8 (C-3), δ 147.1 (C-21, 12), δ 129.6 (C-20, 22, 13, 11), δ 123.7 (C-9, 18), 113.7 (C-14, 10, 19, 23), δ 63.1 (C-5, 2), δ 37.2 (C-17, 8), δ 32.3 (C-7, 16). HRMS m/z (EI⁺) calculated [M+H]⁺353.1972; found: 353.1988.

Synthesis of 3,6-bis(4-((E)-3,3-dimethyltriaz-1-en-1-yl)benzyl)-1,4-dimethylpiperazine-2,5-dione (Compound 1): Compound 2, 1 g, (2.83 mmol) was dissolved in 2.82 ml of 37% HCl (33.96 mmol) and cooled to −5° C. on ice-salt mixture bath. Sodium nitrite, 389 mg (5.64 mmol) was dissolved in 2 ml of water and added dropwise to the solution. The temperature was not allowed to rise above 0 ° C. during addition. After completion of addition, mixture was stirred 10 minutes at 0° C., and added dropwise to the 20 ml (118.90 mmol) of cooled (−5° C.) water solution of dimethylamine (40%). The temperature was kept not more than 0° C. during addition. After completion of addition, mixture was stirred 30 minutes at r.t. and volatiles were evaporated. Obtained liquid was extracted with ethyl acetate, the organic layer was dried over anhydrous sodium sulfate, evaporated, and separated on silica giving Compound 2, 1.05 g (2.26 mmol, 80% yield) as a red crystals. ¹H NMR (400 MHz, CDCl₃) δ ppm: 7.24 (d, J=8.4Hz, 4H), 6.91 (d, J=8.4 Hz, 4H), 3.90 (dd, J=6.6, 3.9Hz, 2H), 3.11 (br. s., 12H), 2.71-2.78 (m, 2H), 2.58 (s, 6H), 2.11 (dd, J=14.3, 6.72Hz, 2H). ¹³C NMR (100 MHz, CDCl₃): δ 164.7 (C-6), δ 164.7 (C-3), δ 149.5 (C-12, 21), ∂ 133.2 (C-9, 18), δ 129.4 (C-14, 10, 19, 23), δ 120.2 (C-20, 22, 11, 13), δ 77.2 (C-28, 33, 32, 34), δ 63.5 (C-5, 2), δ 38.0 (C-8, 17), δ 32.8 (C-16, 7). HRMS m/z (EI⁺) C₂₄H₃₂N₈O₂ calculated: [M+H]⁺465.2721, C₂₂H₂₆N₅O₂⁺392.2081; found: (loses one dimethyltriazene moiety) C₂₂H₂₆N₅O₂⁺392.2082.

### Example 2

### Synthesis of a BBB-Permeable Methylation Conjugate (Compound 19)

High BBB-permeability of N-Me-Phe oligopeptide has been reported by Malakoutikhah et al. on example of the N-Me-Phe-(N-Me-Phe)₃-CONH₂ tetrapeptide. Such oligopeptides allowed transport of various cargos, such as nipecotic acid, 5-aminolevulinic acid, and γ-aminobutyric acid, which by themselves did not show permeability of parallel artificial membrane permeability assays. However, transport across BBB was significantly depended from used payload. For example, the oligopeptides showed high permeability of oligopeptide-nipecotic acid conjugates, while transport of oligopeptide-γ-aminobutyric acid conjugates across the BBB was substantially poorer.

In the conjugate presented herein the BBB-shuttle moiety is modified by introducing dimethyltriazene methylation moiety, creating a conjugate according to some embodiments of the present invention. Such oligopeptides can serve as BBB-permeable shuttles as well as the methylation drug itself. Additionally, other cargoes, such as fluorescent markers and/or other cytotoxic drugs can be attached via N or C-terminus of the oligopeptides.

The synthetic pathway to another BBB-permeable methylation conjugate, comprising an oligopeptide, is presented below. The synthesis starts with the preparation of a building-block unit “alloc protected N-methyl-4-dimethyltriazeno-L-phenylalanine (Compound 9). Compound 9 was prepared from 4-nitro-L-phenylalanine (4-nitro-L-Phe) via the reactions sequence including 11-steps (Scheme 2) with an overall yield of 17.7%. Scheme 2 presents the synthesis of alloc-protected N-methyl-4-dimethyltriazeno-L-phenylalanine (Compound 9)

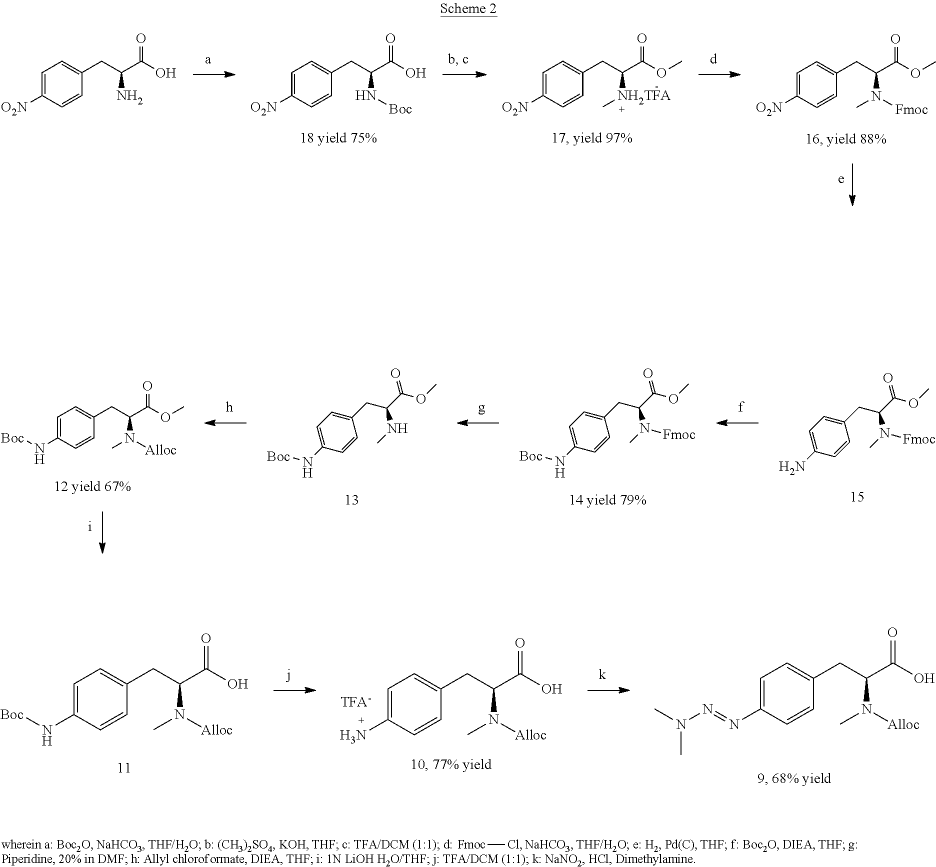

The first step of the reaction sequence was protection of the amino group of 4-nitro-L-Phe by the Boc-protecting group. The choice of this protecting group was based on its stability in basic and lability in acidic conditions. The reaction was leading to 4-nitro-N-Boc-L-phenylalanine (Compound 18) with a yield 75%.

Compound 18 was subjected to methylation (THF, KOH, dimethyl sulfate), followed by Boc deprotection (THF/DCM 1:1) giving pure Compound 17 as a TFA salt with a high yield of 97%. Compound 17 was reacted with Fmoc-chloride (DIPEA, DCM) leading to Fmoc-protected intermediate Compound 16 (88% yield), which was further subjected to reduction (H_{2,} Pd/C, THF) of the nitro group yielding the aromatic amine Compound 15 that was not isolated, and used as it is. Compound 15 was protected by Boc-protecting group to afford Compound 14 (79% yield), and, after successful purification on silica, introduced before the Fmoc-protecting group was switched to Alloc (Fmoc-deprotection: Piperedine/DMF 1:5, to afford Compound 12 that was not isolated; Alloc-protection: Allyl chloroformate, DIPEA, Compound 12, close to 67% yield). Compound 12 was hydrolyzed giving the carboxylic acid Compound 11 that was not isolated as a pure compound, confirmed by TLC/LC/MS) (1N LiOH THF/H_{2O}) and treated with a cold mixture of TFA/DCM leading to Compound 10 (yield close to 77%) that was deposed as a TFA salt in cold ether. The last step included diazotation, followed by reaction with N,N-dimethylamine giving final building-block unit Compound 9 at 68% yield after purification.

Solid phase oligopeptide synthesis was used to afford short oligopeptide using valine and Compound 9 as a building-block units (Scheme 3 below). Taking into account instability of the triazene fragment at low pH' s, the synthesis was performed on 2-chlorotrityl resin, which allows cleavage under relatively mild conditions (3%TFA/DCM). Remarkably, according to the obtained experimental data, the triazene moiety of Compound 9 demonstrates stability at Alloc deprotection conditions (Pd tetrakis, 1,3-dimethylbarbituric acid, 3 hours), as well as under cleavage condition from the resin.

Scheme 3 presents the synthetic pathway of the preparation of triazene-oligopeptide conjugate.

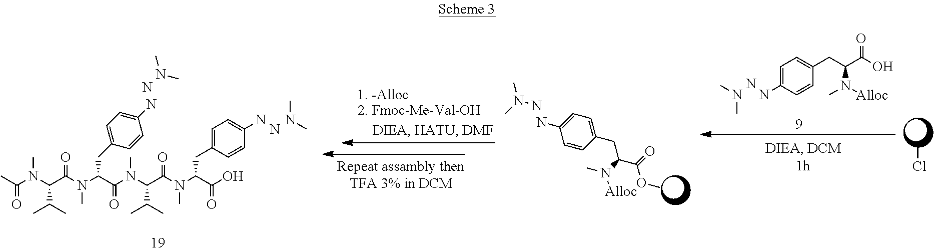

Attaching Compound 9 to the 2-chlorotrityl resin was performed under typical for Solid Phase Oligopeptide Synthesis conditions (DIPEA, DCM, 1.5 hours). After completion of reaction followed by washing with DCM, a small part of the resin was treated with 3% TFA/DCM mixture and analyzed by LC/MS. No additional peaks except of the one allocated to Compound 9 were detected, which confirmed its stability under cleavage conditions. A similar procedure was performed after Alloc deprotection, and the only product found was Alloc-deprotected derivative of the compound.

The second building-block unit, Fmoc-N-Me-Val-OH, was coupled to preloaded Compound 9 using HATU, BTC, PyAOP, PyBroP protocol. After additional cycle and subsequent cleavage from the resin the tetrapeptoid Compound 19 was obtained, representing yet another embodiment of the present invention.

Synthesis of (S)-1-methoxy-N-methyl-3-(4-nitrophenyl)-1-oxopropan-2-aminiurn trifluoroacetate (Compound 17): Compound 18, 10.00 g (32.00 mmol) was dissolved in THF and cooled on ice bath in atmosphere of N_{2.} Potassium hydroxide powder, 18.00 g (0.32 mol) was added portionwise, and stirred 10 minutes at 0° C., followed by dropwise addition of dimethyl sulfate, 15.20 ml (0.16 mol). Reaction progress was controlled by TLC. After completion of reaction, mixture was quenched with ice-citric acid mixture and extracted with ethyl acetate. Organic layer was evaporated, dried over anhydrous sodium sulfate, and treated with cold mixture of TFA/DCM (1:1) during lh. Solvents were evaporated, and cold diethyl ether was added to the residue leading to precipitation of Compound 18. Obtained product was filtered, washed with diethyl ether and dried, giving pure Compound 17 10.91 g (0.031 mol) as the white crystals (yield 97%). ¹H NMR (400 MHz, DMSO-d6) δ ppm: 8.08-8.21 (m, 2H), 7.45-7.56 (m, 2H), 4.66 -4.97 (m, 1H), 3.64-3.74 (m, 3H), 3.27-3.37 (m, 1H), 3.11 -3.25 (m, 1H), 2.55 -2.67 (m, 3H). ¹³C NMR (100 MHz, DMSO-d6): δ 170.9 (C-12), δ 154.1 (C-4), δ 146.4 (C-1), 6 130.7 (C-3), δ 130.7 (C-5), δ 123.4 (C-6), δ 123.4 (C-2), δ 79.6 (C-7), δ 60.8 (C-8), δ 52.3 (C-14), δ 27.8 (C-10). HRMS m/z (EI⁺) C₁₁H₁₄N₂O₄ calculated [M+H]⁺239.1027; found: 239.1034.

Synthesis of (S)-methyl 2-((((9H-fluoren-9-yl)methoxy)carbonyl)(methyl)amino)-3-(4-nitrophenyl)propanoate (Compound 16):

Compound 17 2 g (5.68 mmol) was dissolved in H20/THF mixture (1:1) and cooled on ice bath. Sodium bicarbonate, 2.39 g (28.4 mmol) was added to the mixture followed by stirring 10 minutes at 0° C. Solution of Fmoc-Cl 1.76 g (6.82 mmol) in THF was added dropwise during 30 minutes, mixture was stirred 1 hour, diluted with water and extracted with ethyl acetate. Organic layer was dried over anhydrous sodium sulfate, evaporated, and separated on silica giving Compound 16, 2.3 g (4.50 mmol) 88% yield as colorless oil. ¹H NMR (400 MHz, CDCl₃), mixture of at least two conformers (˜1.5:1) δ: 6.67-8.13 (aromatic protons, m, 12H), 4-77-4.98 (m, 1 H), 4.18-4.54 (m, 3H), 3.76 (s, 1.7H), 3.54 (s, 1.19H), 3.41-3.48 (m, 0.55H), 3.10-3.17 (m, 0.57H), 3.01-3.06 (m, 0.4H), 2.72 (s, 1.55H), 2.66 (s, 1.12H), 2.56-2.63 (m, 0.45H). ¹³C NMR (100 MHz, CDCl3): δ170.7 (C-28) major, 170.0 (C-28) minor; 157.1 (C-16) major, 156.4 (C-16) minor; 147.1 (C-25) major, 146.9 (C-25) minor; 144.8 (C-2, 5) major, 144.6 (C-2, 5) minor; 143.7 (C-3, 4) minor, 143.5 (C-3, 4) major; 141.6 (C-22) major, 141.4 (C-22) minor; 129.8 (C-10) major, 129.6 (C-10) minor; 128.0 (C-26, 24) minor, 127.9 (C-26, 24) major; 127.3 (C-11, 7) minor, 127.1 (C-11, 7) major; 124.8 (C-8, 12) major, 124.4 (C-8, 12) minor; 129.9 (C-9, 13) major, 123.8 (C-9,13) minor; 120.2 (C-27, 23) minor, 120.1 (C-27, 23) major; 68.2 (C-30) major, 67.4 (C-30) minor; 60.3 (C-14) minor, 60.2 (C-14) major; 52.8 (C-20) major, 52.7 (C-20) minor; 47.2 (C-1) minor, 47.1 (C-1) major; 34.8 (C-21) major, 34.7 (C-21) minor; 32.7 (C-19) minor; 32.0 (C-19) major. HRMS m/z (EI⁺) C26H24N206 calculated [M+H]⁺461.1707; found: 461.1736.

Synthesis of (S)-methyl 2-((((9H-fluoren-9-yl)methoxy)carbonyl)(methyl)amino)-3-(4-((tert-butoxycarbonyl)amino)phenyl)propanoate (Compound 14): Compound 16, 2 g (4.30 mmol) was dissolved in THF, 10% Pd/C 0.3 g was added and mixture was placed in hydrogenator (2 atm, 2 hours, room temp.). After completion of reduction solution was filtered from the carbon and mixed with 0.94 g (4.30 mmol) of Boc anhydride and DIPEA 1.5 ml (8.6 mmol). Completion of reaction was monitored by TLC and LC/MS. When the starting material was consumed, ice water was added, and mixture was extracted with ethyl acetate. Organic layer was dried over anhydrous sodium sulfate, evaporated, and purified on silica giving Compound 14 1.8 g (3.40 mmol) 79% yield as the light-yellow oil. ¹H NMR (400 MHz, CDCl3), mixture of at least two conformers (˜1.8:1) δ: 6.36-7.77 (aromatic protons, m, 12H), 4.14-4.99 (m, 4H), 3.74 (s, 1.9H), 3.61 (s, 1.03H), 3.31 (dd, j=14.6, 5.4; 0.55H), 2.97-3.10 (m 1H), 2.81 (s, 1.66H), 2.77 (s, 1.02H), 2.63-2.71 (m 0.55H), 1.5 (m 9H). ¹³C NMR (100 MHz, CDCl₃): δ 171.4 (C-28) major, 170.9 (C-28) minor; 156.5 (C-16), 155.9 (C-16) minor; 152.6 (C-32) major, 152.2 (C-32) minor; 144.0 (C-25) major, 143.8 (C-25) minor; 141.4 (C-22) minor, 141.3 (C-22) major; 137.0 (C-4, 3); 131.5 (C-7, 11); 129.3 (C-6, 10), major, 129.3 (C-6, 10 minor); 127.6 (C-27, 23) minor, 127.6 (C-27, 23) major; 127.0 (C-26, 24); 125.0 (C-5, 2) major, 124.7 (C-5, 2) minor; 120.0 (C-8, 12) major, 119.9 (C-8, 12) minor; 118.6 (C-9, 13) 80.4 (C-34); 67.7 (C-14) major, 67.3 (C-14) minor; 60.3 (C-30) minor, 60.2 (C-30) major; 52.3 (C-20) major, 52.2 (C-20) minor; 47.2 (C-10) minor, 47.1 (C-10) major; 34.4 (C-21) minor, 34.3 (C-21) major; 31.8 (C-19) minor, 31.6 (C-19) major; 28.3 (C-39, 35, 38). HRMS m/z (EI⁺) C₃₁H₃₄N₂O₆ calculated [M+H]⁺531.2490; found 531.2511.

Synthesis of (S)-methyl 2-(((allyloxy)carbonyl)(methyl)amino)-3-(4-((tert-butoxycarbonyl)amino)phenyl)propanoate (Compound 12): Compound 14 1.5 g (2.83 mmol) was stirred dissolved in 20 ml of mixture Piperedine/DMF (1:5) and stirred 30 minutes (reaction was monitored by LC/MS. Liquids were evaporated, residue was dissolved in THF, cooled on ice bath under N₂ atmosphere. DIPEA, 986 μL (5.66 mmol) was added followed by dropwise addition of allyl chloroformate 601.6 μL (5.66 mmol). The progress of reaction was monitored by LC/MS. After completion of reaction, mixture was diluted with cold 1 M citric acid solution end extracted with ethyl acetate. The organic layer was dried over anhydrous sodium sulfate, evaporated and purified on silica, giving Compound 12, 0.74 g (1.90 mmol) as yellow oil, 67% yield. ¹H NMR (400 MHz, CDCl3) mixture of conformers, δ ppm: 7.21-7.35 (m, 2H), 7.03-7.17 (m, 2H), 6.60 (br. s., 1H), 5.69-6.03 (m, 1H), 5.13-5.26 (m, 2H), 4.73-4.97 (m, 1H), 4.46-4.60 (m, 2H), 3.67-3.79 (m, 3H), 3.20-3.35 (m, 1H), 2.90-3.04 (m, 1H), 2.57-2.90 (m, 3H), 1.36-1.64 (m, 9H). ¹³C NMR (100 MHz, CDCl₃): δ171.5 (C-19) major, 171.2 (C-19) minor; 156.7 (C-22) major, 155.8 (C-22) minor; 153.1 (C-8); 137.0 (C-1) minor, 136.9 (C-1) major; 132.7 (C-25) major, 132.5 (C-25) minor; 131.6 (C-4); 129.4 (C-2, 6) minor, 129.3 (C-2, 6) major; 118.8 (C-5, 3); 117.5 (C-26) minor, 117.1 (C-26) major; 80.7 (C-10); 66.4 (C-24) minor, 66.3 (C-24) major; 60.7 (C-21) minor, 60.1 (C-21) major; 52.3 (C-13) minor, 52.3 (C-13) major; 34.7 (C-12) minor, 34.2 (C-13) major; 32.2 (C-15) minor, 31.5 (C-15) major; 28.3 (C-18, 11, 17). HRMS m/z (EI⁺) C₂₀H₂₈N₂O₆ calculated [M+H]+393.2020; found: 393.2032.

Synthesis of (S)-4-(2-(((allyloxy)carbonyl)(methyl)amino)-2-carboxyethyl)benzenaminium trifluoroacetate (Compound 11): Compound 12 0.7 g (1.78 mmol) was dissolved in 1N LiOH solution (THF/H2O) and stirred 1 hour. The completion of ester hydrolysis was controlled by TLC. The mixture was acidified to pH 5 with cold citric acid and extracted with ethyl acetate. The organic layer was dried over anhydrous sodium sulfate, evaporated and subjected to Boc deprotection by cold TFA/DCM (1:1) mixture during 1 hour. The solvents were evaporated, and cold diethyl ether was added to the residue forming brown oil of crude product. Purification on silica led to pure Compound 11 0.54 g (1.37 mmol) as a brown oil, yield 77%. ¹H NMR (400 MHz, CD₃CN): was obtained as the mixture of two isomers. Isomer A (cis): Ar, 2H meta δ 7.19; Ar, 2H ortho 7.23; CH, 1H δ 5.81; CH₂, 2H δ 5.12; CH, 1H δ 4.80; CH₂, 2H δ 4.43; CH₂, 1H δ 3.23, 1H δ 3.07; CH_{3,} s 3H, δ 2.73. ¹³C NMR (100 MHz, CD3CN): —COOH, δ 173.1; CO carbamate, δ 157,5; Ar C—CH_{2,} ipso δ 138.2; allylic C, δ 134.0; Ar C—NH2, ipso δ 132.6; Ar C, ortho δ 131.2; Ar C, meta δ 123.0; allylic C (gem H), δ 117.3; allylic CH₂, δ 66.8; CH, δ 61,4; benzylic CH₂, δ 34.6; N(CH₃) δ 32.6. Isomer B (trans): ¹H NMR (400 MHz, CD₃CN): Ar, 2H meta δ 7.19; Ar, 2H ortho 7.23; CH, 1H δ 5.81; CH₂, 2H δ 5.12; CH, 1H δ 4.80; CH₂, 2H δ 4.43; CH₂, 1H δ 3.23, 1H δ 3.07; CH_{3,} s 3H, δ 2.69. ¹³C NMR (100 MHz, CD₃CN): —COOH, δ 172.8; CO carbamate, δ 156,8; Ar C, ipso C—CH₂δ 138.1; allylic C, δ 133.9; Ar C—NH₂ ipso, δ 132.7; Ar C, ortho δ 131.3; Ar C, meta δ 123.0; allylic C (gem H), δ 117.6; allylic CH₂, δ 66.8; CH₂, δ 61,1; benzylic CH₂, δ 35.0; N(CH₃) δ 32.5.HRMS m/z (EI⁺) C₁₅H₂₀N₂O₄ calculated [M+H]⁺293.1496; found: 293.1502.

Synthesis of (S,E)-2-(((allyloxy)carbonyl)(methyl)amino)-3-(4-(3,3-dimethyltriaz-1-en-1-yl)phenyl)propanoic acid (Compound 9): Compound 10, 0.5 g (1.71 mmol) was dissolved in 850 μL of HCl (37%) and cooled to −5° C. on ice-salt bath. Sodium nitrite, 118.00 mg (1.71 mmol) was dissolved in 1 ml of water and added dropwise to the solution. The temperature was not allowed to rise above 0° C. during addition. After completion of addition, mixture was stirred 10 minutes at 0° C., and added dropwise to the 20 ml (118.90 mmol) of cooled (−5-0° C.) water solution of dimethylamine (40%). The temperature was kept below 0° C. during addition. After completion of addition, mixture was stirred 30 minutes at r.t. and volatiles were evaporated. Acetonitrile was added to the residue, and obtained mixture was purified on preparative HPLC using water/acetonitrile with addition of 0.1% of NH₄OH. After lyophilization, Compound 9 388.38 mg (1.16 mmol, 68% yield) was obtained as dark-yellow crystals. ¹H NMR (400 MHz, DMSO-d6) was obtained as the mixture of two isomers. Isomer A (cis): Ar, 2H meta δ 7.15; Ar, 2H ortho 7.22; CH, 1H δ 5.82; CH₂ , 2H δ 5.13; CH, 1H δ 4.69; CH₂, 2H δ 4.40; CH₂, 1H δ 3.23, 1H δ 2.84; CH₃, 3H, s δ2.74; 6H, s δ 3.28. ¹³C NMR (100 MHz, DMSO-d6): —COOH, δ 173.0; CO carbamate, δ 155,9; Ar C—N, ipso δ 148.6; Ar C—CH₂, ipso δ 136.2; allylic C, δ 133.4; Ar C, meta δ 129.0; Ar C, ortho δ 119.8; allylic C (gem H), δ 116.3; allylic CH₂, δ 64.4; CH, δ 61,0; benzylic CH₂, δ 34.6; dimethyl (triazene moiety) two broad peaks at δ 42 and δ 36; N(CH₃) δ 30.51. Isomer B (trans): ¹H NMR (400 MHz, DMSO-d6): Ar, 2H meta δ 7.15; Ar, 2H ortho 7.22; CH, 1H δ 5.70; CH₂, 2H δ 5.09; CH, 1H δ 4.63; CH₂, 2H δ 4.32; CH₂, 1H δ 3.23, 1H δ 2.84; CH₃, 3H, s δ 2.72; 6H, s δ 3.28. ¹³C NMR (100 MHz, DMSO-d6): —COOH, δ 172.8; CO carbamate, δ 155,6; Ar C—N, ipso δ 148.6; Ar C—CH₂, ipso δ 136.2; allylic C, δ 133.5; Ar C, meta δ 129.1; Ar C, ortho δ 119.8; allylic C (gem H), δ 116.1; allylic CH₂, δ 64.7; CH, δ 61,3; benzylic CH₂, δ 34.9; dimethyl (triazene moiety) two broad peaks at δ 42 and δ 36; N(CH₃) δ 30.8.HRMS m/z (EI⁺) C₁₆H₂₂N₄O₄ calculated [M+H]+335.1714; found 335.1738.

### Example 3

### Characterization of BBB-Permeable Methylation Conjugates

Chemo-Stability:

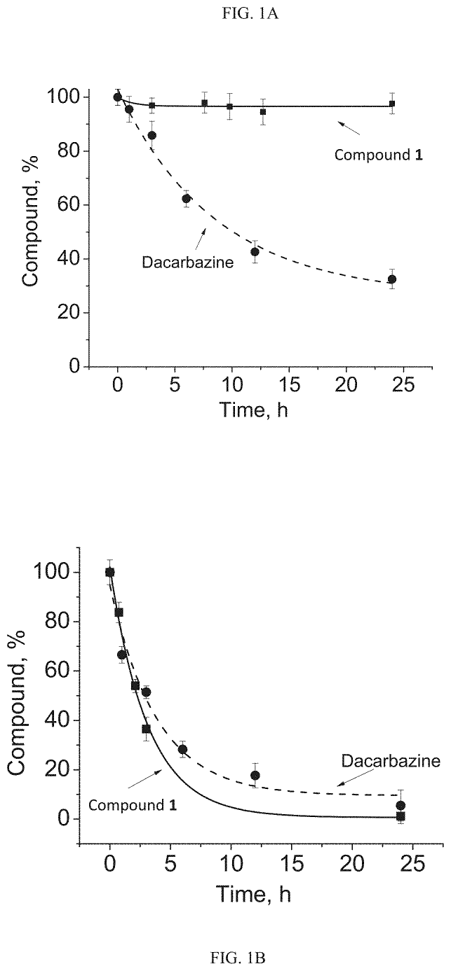

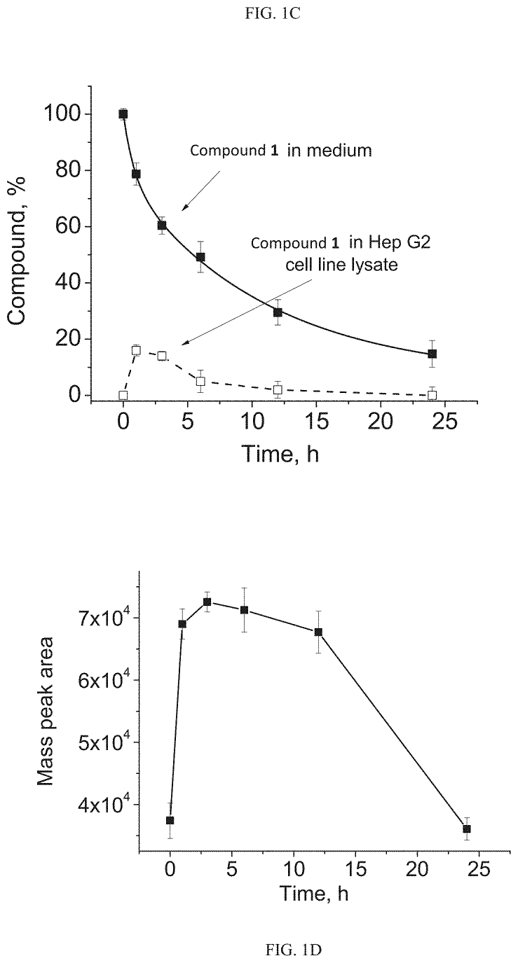

These studies were performed for Compound 1 at two pH values: 4.6 and 7.4, and compared to triazene drug dacarbazine (DTIC). These chosen pHs mimic relatively acidic cancer intracellular (pH 4.6) and slightly basic physiological (pH 7.4) environments. Aliquots taken at selected time intervals were analyzed by LC-MS, and the results are shown in FIGS. 1A-D.

FIGS. 1A-D present the results of stability and degradation assays conducted for Compound 1 in PBS (FIG. 1A), acetate buffer (FIG. 1B), Hep G2 cell line (FIG. 1C): medium vesus cell lysate, and the detection of the metabolite (E)-3-(4-aminobenzyl)-1,4-dimethyl-6-(4-(3-methyltriaz-1-en-1-yl)benzyl)piperazine-2,5-dione (see, scheme below) in Hep G2 cell line lysate by measurement of mass peak area (LCMS) (FIG. 1D).

As can be seen in FIGS. 1 A-D, the stability of measured Compound 1 varied depending on pH. In a PBS buffer (pH 7.4, FIG. 1A) Compound 1 exhibited sufficient stability compared to DTIC (t_{1/2}=10.2 h). However, in acidic pH 4.6 (FIG. 1B) both Compound 1 and DTIC were similarly unstable (t_{1/2}=4.8 and h t_{1/2}=5.1 hours, respectively). The results for DTIC are consistent with the reported stability of the control drug in various hydrolytic media. In addition, Compound 1 decomposition has been studied by hepatic cytochrome P450 induction in hepatocellular carcinoma HepG2 cells using LC-MS (FIG. 1D). HepG2 cells displayed similarity to human hepatocytes regarding the expression of cellular proteins. The decomposition of Compound 1 was examined in cell medium and in cell lysate, while the identification of metabolites was carried out solely in cell lysate. During the incubation in cell medium we observed continuous decomposition of Compound 1 (FIG. 1C). However, in cell lysate Compound 1 has gradually accumulated within an hour of incubation and decomposed subsequently (FIG. 1C). In the course of the search after possible metabolites in cell lysate, the main metabolite shown in the scheme below that has been detected ([M+H]⁺=395.2) containing monomethyl triazene (MTIC like) and aniline (AIC like) groups (FIG. 1D).

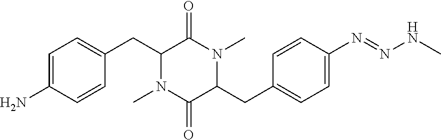 [SMILES: CN/N=N/c1ccc(CC2C(=O)N(C)C(Cc3ccc(N)cc3)C(=O)N2C)cc1]

(E)-3-(4-aminobenzyl)-1,4-dimethyl-6-(4-(3-methyltriaz-1-en-1-yl)benzyl)piperazine-2,5-dione
([M+H]+=395.2)

The above metabolite exhibited detectible accumulation/decomposition plot by calculating the area under the MS peak of each tested sample respectively (FIG. 1D). It is assumed that this metabolite is the result of oxidation of Compound 1 by hepatic cytochrome P450 and subsequent degradation of two dimethyl triazene tethers in agreement with the mechanism of action of dacarbazine. The observed stability of Compound 1 in PBS potentiates its pharmacological utility during circulation in blood. Therefore, it is concluded that the stability of Compound 1 is sufficient for proceeding to animal models.

In-vivo activity:

The efficacy of Compound 1 in inhibition of intracranial malignant brain glioblastoma growth and survival in nude mice was evaluated using nude mice that were implanted with 5×10⁵ A172 GBM cells in the right striatum, followed by consequent once daily five intraperitoneal injections with the vehicle, TMZ (25 mg/kg) and Compound 1 (50 mg/kg), on the tenth day after glioblastoma implantation. The mice handling and all procedures performed on the animals were reviewed and approved by the Institutional Animal Care and Use Committee of Ariel University before the initiation of the study, approval N° IL-146-11-17.

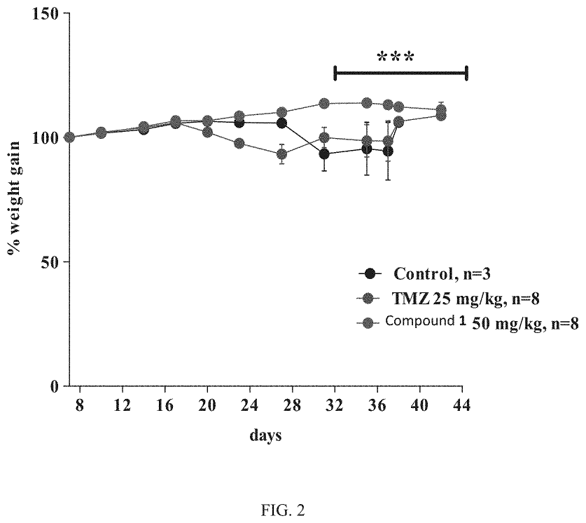

FIG. 2 presents intracranial tumor-bearing mice weight gain, wherein the plot of the mice treated with Compound 1 is marked with red circles, plot of mice treated with TMZ is marked with blue circles, and plot of the control group is marked by black circles (GraphPad Prism 5; and “***” p<0.001).

As can be seen in FIG. 2, the mice treated with Compound 1 demonstrated normal weight gain compared with the vehicle treated and TMZ treated mice. Significant body weight gain reduction was measured in TMZ and vehicle treated mice using repetitive two way ANOVA with Bonferroni post-test. Tumor bearing mice body weight and monitoring of clinical signs revealed the beneficial effect of treatment with Compound 1 compared with TMZ treatment's biphasic effect (weight drop and weight recovery) and vehicle treatment worsened the effect in mice. Compound 1 treatment demonstrated a prominent effect on tumor bearing mice survival rate. Mice brains were collected at termination day determined by appearance of severe clinical signs within tumor progression and invasion in brain tissue. Brains were fixed with 4% PFA, paraffin embedded and serially cut in to 5 μm. Three slides within a distance of 100 μm apart were sampled for each brain, and stained with hematoxyline-eosin dyes (H&E). Tumor area, distribution and invasion in to brain tissue were observed under light microscopy.

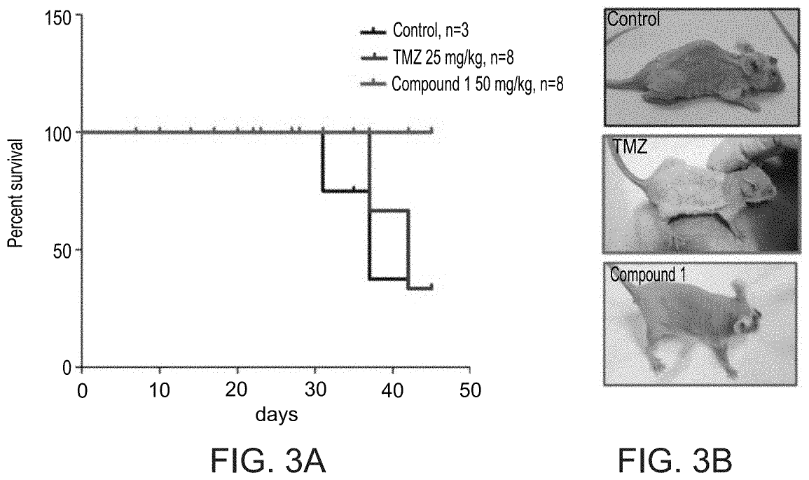

FIGS. 3A-B present the results obtained in the survival rate assays (FIG. 3A), and mice appearance on termination day (FIG. 3B.).

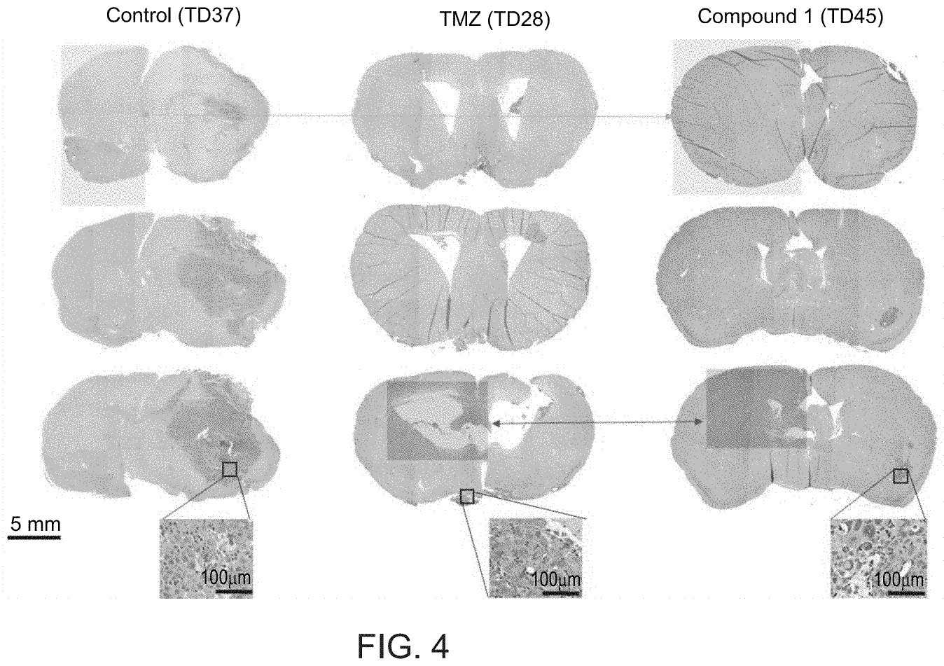

As can be seen in FIG. 3A, noticeable reduction in tumor area, tumor distribution and invasion were observed in brain sections derived from TMZ and Compound 1 treated mice. However, significant brain structure distortion was noticed in brain sections derived from control animals (see, FIG. 4) due to extensive tumor growth and compression of the healthy hemisphere. In the brain sections of TMZ treated mice, both lateral and third ventricles were significantly enlarged, evidencing CSF retention and appearance of hydrocephalus pathology. However, normal brain structure was noticed in brain sections of Chimera treated mice (see, FIG. 4).

FIG. 4 presents GBM tumor presence and distribution in brain sections stained with H&E, wherein three slides within a distance of 100 μm apart represent one mice from each experimental group.

In summary, Compound 1, a conjugate according to some embodiments of the present invention, provided a beneficial therapeutic effect for GBM treatment, as shown in an animal model study using mice intracranial GBM model. Mice treated with Compound 1 demonstrated good tolerability to treatment in comparison to the TMZ drug, which was relatively toxic and poorly tolerable, reflected by body weight and survival decrease in mice bearing GBM tumors. Further investigation should be performed in order to determine best dose and safety. The examples presented hereinabove demonstrated the efficacy of Compound 1 in the BGM mouse model using the TMZ sensitive cancer cell lines. Further investigation should pay attention to GBM TMZ resistant lines and to TMZ resistance development during the treatments. Due to their good tolerability, this exemplary DKP-based BBB-permeable methylation conjugate opens up the possibility to overcome the formidable obstacle of the BBB, increasing the accumulation of therapeutic agent Me+ at the target, thereby achieving efficient drug delivery to the brain.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A compound comprising:
a blood-brain barrier shuttle moiety, and
at least two methylation moieties, each independently represented by general Formula I: 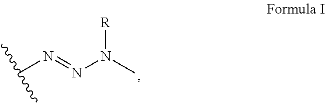
wherein R is selected from the group consisting of hydrogen, a C₁₋₄ alkyl, a labeling moiety, and a bioactive agent.

2. The compound of claim 1, wherein said blood-brain barrier shuttle moiety is a small molecule selected from the group consisting of 1,4-dialkyl-3,6-diarylpiperazine-2,5-dione, 1,4-dimethyl-3,6-diarylpiperazine-2,5-dione (DKP), diaza diketocyclooctane, and N,N′-diaza diketocyclooctane.

3. The compound of claim 2, represented by general Formula II:

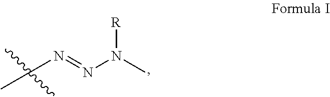

4. The compound of claim 3, being (3S,6S)-1,4-dimethyl-3,6-bis(4-((E)-3-methyltriaz-1-en-1-yl)benzyl)piperazine-2,5-dione or (3S,6S)-3,6-bis(4-((E)-3,3-dimethyltriaz-1-en-1-yl)benzyl)-1,4-dimethylpiperazine-2,5-dione (Compound 1).

5. The compound of claim 1, wherein said blood-brain barrier shuttle moiety is an oligopeptide selected from the group consisting of (N-Me-Phenylalanine)ₙ, (N-Me-Tryptophan)ₙ, (N-Me-1-Naphthylalanine)ₙ, (N-Me-2-Naphthylalanine)ₙ, (N-Me-Tyrosine)ₙ and (N-Me-DOPA)ₙ, whereas n is an integer equal or greater than 2.

6. The compound of claim 5, wherein at least one of said at least two methylation moieties is attached to a side-chain of at least one of said amino-acids of said oligopeptide.

7. A pharmaceutical composition comprising, as active ingredients, the compound of claim 1, and a pharmaceutically acceptable carrier or excipient.

8. The composition of claim 7, being packaged in a packaging material and identified in print, in or on said packaging material, for use in the treatment of cancer.

9. The composition of claim 8, being packaged in a packaging material and identified in print, in or on said packaging material, for use in diminishing brain tumors.

10. A compound comprising an amino acid and a methylation moiety attached to a side-chain of the amino acid, represented by general Formula III: 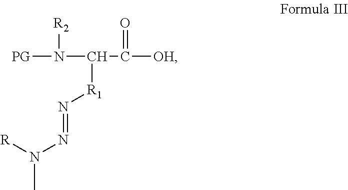
wherein:
R is selected from the group consisting of hydrogen, a C₁₋₄ alkyl, a labeling moiety, and a bioactive agent,
R₁ is said side-chain,
R₂ is a hydrogen or a C₁₋₄ alkyl, and
PG is hydrogen or a protecting group.

11. The compound of claim 10, wherein R₁ is selected from the group consisting of a branched or unbranched, substituted or unsubstituted alkyl interrupted or uninterrupted by one or more heteroatom, a substituted or unsubstituted aryl, and a substituted or unsubstituted heteroaryl.

12. The compound of claim 11, wherein R₁ is a p-, m- or o-substituted phenyl, R₂ is hydrogen, methyl or ethyl, R is hydrogen or methyl, and PG is hydrogen or an α-amino protecting group.

13. The compound of claim 10, being Compound 9 as set forth hereinabove.

14. The compound of claim 10, wherein said labeling moiety or said bioactive agent is attached to said methylation moiety via a labile linking moiety.

15. An oligopeptide comprising at least one building-block unit derived from the compound of claim 10.

16. The oligopeptide of claim 15, being (2R,5S,8R,11S)-2,8-bis(4-((E)-3,3-dimethyltriaz-1-en-1-yl)benzyl)-5,11-diisopropyl-3,6,9,12-tetramethyl-4,7,10,13-tetraoxo-3,6,9,12-tetraazatetradecanoic acid (Compound 19).
